(19) United States
(12) Patent Application Publication
(10) Pub. No.: US 2008/0077055 A1
(43) Pub. Date: Mar. 27, 2008

(54) Home suggested immobilization test (SIT) monitor and methodology

(75) Inventor: Robert Allen, Arnold, MD (US)

Correspondence Address: LARRY J. GUFFEY, WORLD TRADE CENER - SUITE 1800, 401 EAST PRATT STREET, BALTIMORE, MD, 21202, US

(73) Assignee: MANESS-ALLEN LLC, Arnold, MD (US)

(21) Appl. No.: 11/899,536

(22) Filed: Sep. 6, 2007

Related U.S. Application Data
(60) Provisional application No. 60/842,635, filed on Sep. 6, 2006.

(51) Int. Cl. A61B 5/11

(52) U.S. Cl. 600/595

(57) ABSTRACT

A Restless Legs Syndrome (RLS) sensory symptoms monitoring device for recording and analyzing, with the use of a personal computer, the RLS sensory symptoms of one wearing the device during a specified test period in which the wearer complies with a given test protocol includes: (a) a switch for the wearer to input, at prescribed time intervals during the test period, the wearer's RLS sensory symptoms assessment, (b) an alarm for indicating to the wearer the instances of the prescribed times during the period when the assessments are to be made and inputted into the device, (c) a sensor that measures the compliance of the wearer with the test protocol, (d) a microprocessor having embedded programmable memory, (e) a storage element for the inputted assessments and data, and (f) firmware for controlling the microprocessor's operation.

### CROSS-REFERENCE TO RELATED APPLICATION

This application claims the benefit of U.S. Provisional Patent Application No. 60/842,635, filed Sep. 6, 2006 by the present inventor.

### BACKGROUND OF THE INVENTION

1. Field of the Invention

The present invention generally relates to an apparatus and methods for monitoring and recording a patient's experience of Restless Legs Syndrome (RLS) sensory symptoms. This information is useful in diagnosing and evaluating the severity of the RLS condition in a patient.

2. Background for Development of the Present Invention

Restless Legs Syndrome (RLS) is a sensory disorder whose symptoms include experiencing abnormal sensations of a compelling urge to move one's legs. It is usually, but not always, associated with dysesthesias (i.e., tactile hallucinations such as peculiar moving sensations deep in the legs). These symptoms are relieved almost immediately and persistently by walking or other physical activity.

Two common misperceptions have developed about RLS. First, RLS is falsely seen primarily as a movement (periodic limb movements in sleep (PLMS) are commonly seen in RLS patients) rather than a sensory disorder, and second, RLS is equally falsely seen as a minor disorder of little clinical significance. The clinical significance of RLS however can no longer be denied.

RLS occurs in about 7% of adults in the United States and Europe with a wide spectrum of severity ranging from occasionally annoying to severely disruptive of normal life function. The clinically-significant, moderate-to-severe RLS occurs in about 2-3% of the American and European population and reduces quality of life as much or more than other chronic medical conditions such as heart failure and depression. Severe RLS can disrupt all aspects of a sufferer's life and can produce the most profound sleep loss of any chronic medical disorder.

Despite the impact of RLS, the methods and standards for the quantification of its symptoms remain regrettably nascent. These include:

Clinical Scales:

Several clinical scales have been developed. They range from a global review of the severity of various symptom characteristics over a specified time period (one to two weeks) to questions about overall symptom severity in relation to specific activities and time-of-day. The best-validated and most commonly used scale is that developed by the international restless legs syndrome study group referred to as the IRLS (international restless legs scale). This scale asks about the severity of specific aspects or effects of symptoms as they occurred over the past week (or past fortnight). The IRLS has been shown to correlate reasonably well with sleep laboratory evaluations of the sleep disturbance produced by RLS. However, this scale has also been seen to be significantly influenced by placebos (e.g., about 73% of the active treatment effect).

Another scale of note, the RLS-6, includes questions relating severity reports to activity level and time-of-day, but otherwise takes the same global recall approach used in the IRLS.

RLS—Sleep Logs:

These have been developed to allow a patient or subject to record during the day the occurrence of symptoms and also their sleep-wake state. If the subject follows instructions, such a log reduces the recall bias and permits assessment over several days to assess daily variation in symptoms.

The problems with these logs generally involve the lack of patient compliance, including a tendency to complete them shortly before the time they are reviewed rather than at the time symptoms occur. Electronic forms of these are available, but the frequent disturbance during the day to obtain this information complicates acceptance and use.

Periodic Leg Movement (PLM) Assessments:

Periodic leg movements (PLM), both in sleep (PLMS) and wake (PLMW), have been used to provide an objective assessment of RLS. These represent a non-specific, motor sign of RLS and appear to provide a seemingly objective assessment of RLS severity (i.e., little or no placebo effects seen in clinical trials).

However, there are two significant problems with using these PLM measures. The first is the expense and inconvenience due to the need to conduct such assessments in sleep laboratories (e.g., where electroencephalograms (EEGs (electrical activity of the brain) are used to separate sleep and waking states). The second and somewhat more significant problem is that about 15-20% of RLS patients do not have a significant number of PLMS during the night. The failure to have a significant number of PLMS (PLMS/hr >5) makes it difficult to see improvements with treatment.

Laboratory Suggested Immobilization Test (SIT):

A much different approach to evaluating RLS severity focuses on standardizing the conditions that provoke symptoms. In this laboratory test, patients have their EEG and leg electromyograms (i.e., bilateral anterior tibialis EMG (electrical activity of a muscle)) recorded while they are sitting up at a 45 degree angle in bed with their feet stretched out horizontally and having been told to avoid moving voluntarily. The test lasts sixty minutes during which time there are to be no distracting stimuli in the room with the patient. Every five minutes during the SIT test the patient is asked to indicate, using a moving pointer on a vertical scale, the level of his leg discomfort, the primary sensory symptom of RLS.

From reviewing these current test methods, it seems apparent that the focus of attention for diagnosing and evaluating RLS needs to shift to a better evaluation of RLS′ sensory symptoms (e.g., evaluations of sensory symptoms under standard conditions that are known to provoke the disorder and/or over several days (3-5) to obtain a more valid measure of the disease status and severity). There exists a need to develop methods for evaluating the sensory symptoms of RLS that minimize the impact of those reported factors that complicate the clinical evaluation of the sensory symptoms of RLS and contribute to the limitations of existing measures for evaluating these symptoms.

3. Objects and Advantages

There has been summarized above, rather broadly, the background that is related to the present invention in order that the context of the present invention may be better understood and appreciated. In this regard, it is instructive to also consider the objects and advantages of the present invention.

It is an object of the present invention to provide both methods and an apparatus for monitoring and recording a patient's experience of Restless Legs Syndrome (RLS) sensory symptoms so as to aid in diagnosing and evaluating the severity of this condition in a patient.

It is also an object of the present invention to develop monitoring methods and apparatus that address and minimize the impact of those factors or considerations that complicate clinical evaluations of the sensory symptoms of RLS.

It is a further object of the present invention to provide a RLS symptoms monitoring test that: 1) uses duration of a standardized rest procedure to provoke symptoms thereby both reducing variable effects of activity masking the symptoms and also stimulating maximum expression of symptoms under a standardized condition, 2) permits repeated assessment over a number of days (e.g., 5 days) to reduce effects of day-to-day variations, 3) documents the subjective report of sensory symptoms as they are occurring at the time of near maximum severity to both seek full expression of the symptoms and mitigate the problem of recall in the clinic setting when the symptoms may be minimal, if present at all, and 4) provides for a simultaneous recording of PLM that can be used as a motor sign measure to support RLS assessments.

It is an object of the present invention to provide both means and methods to measure the sensory symptoms of RLS under standardized conditions in the home environment using a technology that permits unattended monitoring without involvement of any technical staff.

It is additionally an object of the present invention to provide both means and methods to measure the sensory symptoms of RLS that will permit one to obtain a concomitant assessment of leg movements that can be matched for time of occurrence with RLS′ sensory symptoms.

It is also an object of the present invention to provide a standard method for diagnosing RLS and its severity; a method that would be used not only for research studies, but also for routine clinical practice in major centers that treat RLS and in clinical trials for treatment evaluations.

These and other objects and advantages of the present invention will become readily apparent as the invention is better understood by reference to the accompanying summary, drawings and the detailed description that follows.

### SUMMARY OF THE INVENTION

Recognizing the need for improved devices and methods for monitoring and recording a patient's RLS sensory symptoms, the present invention is generally directed to satisfying the needs set forth above and the problems identified with prior systems for assessing RLS.

In accordance with one preferred embodiment of the present invention, the foregoing need can be satisfied by providing a RLS sensory symptoms monitoring device for recording and analyzing, with the use of a personal computer, the RLS sensory symptoms of one wearing the device during a specified test period in which the wearer complies with a given test protocol (e.g., sitting up at a 45 degree angle in bed with feet stretched out horizontally) The device includes: (a) a means (e.g., a switch) for the wearer to input, at prescribed time intervals during the test period, into the device the wearer's assessment of the wearer's RLS sensory symptoms, (b) a means (e.g., a visual or audio alarm) for indicating to the wearer the instances of the prescribed times during the period when the assessments are to be made and inputted into the device, (c) a means that measures the compliance of the wearer with the test protocol (e.g., a position or motion sensor attached to the individual's ankle or wrist), (d) a means that measures a motor sign (e.g., PLM) of the wearer that can provide a more objective (i.e., non-sensory) assessment of RLS phenomena, (e) a microprocessor having embedded programmable memory, (f) a storage means for the inputted assessments and data, and (g) firmware for controlling the microprocessor's operation.

In accordance with another preferred embodiment, the present invention takes the form of the methodology or method for performing “Sensory Symptoms During Home-Based Self-Imposed Rest Evaluator” (hereinafter denoted as a SHIRE) testing for diagnosing and evaluating the severity of a patient's RLS condition. This method includes the steps of: (a) imposing on the patient during the test a given test protocol, (b) utilizing a SHIRE monitor into which is inputted the patient's assessment at prescribed time intervals of his/her RLS sensory symptoms, (c) indicating to the patient the instances of prescribed times during the period when the assessments are to be made and inputted, (d) sensing and inputting into the device data that measures the wearer's compliance with the test protocol (e.g., monitoring of wearer's position, degree of activity and surrounding environmental conditions) and a motor sign from the wearer that can serve as a more objective measure of the RLS phenomena experienced by the wearer, (e) utilizing a microprocessor within the device to control these indication signals, sensing and the storage of data and assessments, and (f) transferring the stored assessments and data to a personal computer for analysis.

Thus, there has been summarized above, rather broadly and understanding that there are other preferred embodiments which have not been summarized above, the present invention in order that the detailed description that follows may be better understood and appreciated. There are, of course, additional features of the invention that will be described hereinafter and which will form the subject matter of the later presented claims to this invention.

### BRIEF DESCRIPTION OF THE DRAWINGS

FIG. 1 illustrates the present invention in the form of a “Sensory Symptoms During Home-Based Self-Imposed Rest Evaluator (hereinafter denoted as a SHIRE monitor or monitoring device)”.

FIG. 2 is a schematic diagram of a preferred embodiment of the present invention.

### DESCRIPTION OF THE PREFERRED EMBODIMENT

Before explaining at least one embodiment of the present invention in detail, it is to be understood that the invention is not limited in its application to the details of construction and to the arrangements of the components set forth in the following description or illustrated in the drawings. Also, it is to be understood that the phraseology and terminology employed herein are for the purpose of description and should not be regarded as limiting.

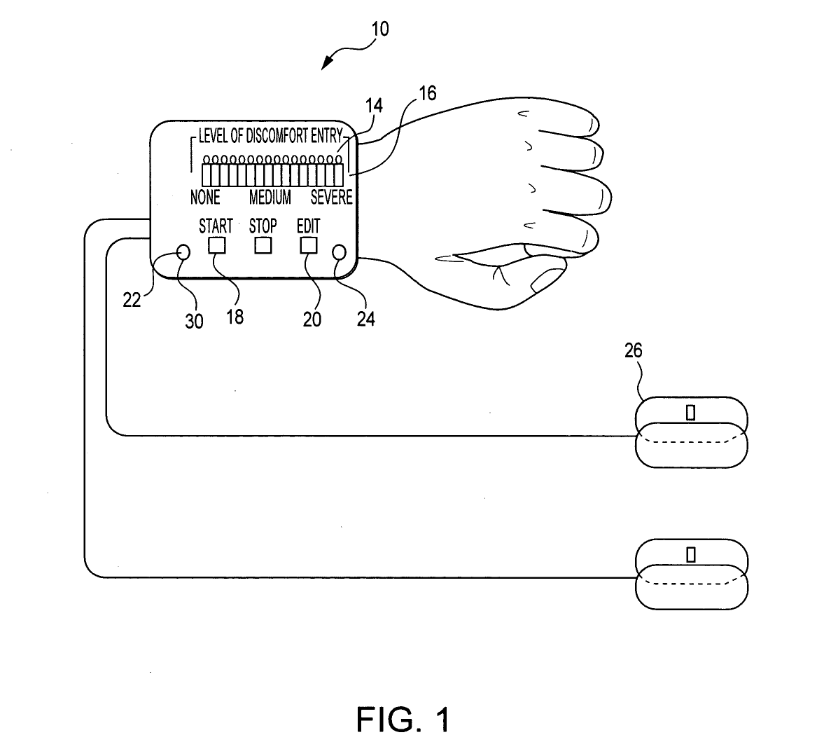

A preferred embodiment of the present invention, in the form of a SHIRE monitor or device, consists in general of: (i) a handheld, battery powered, microcontroller based device for recording and storing a patient or user's ratings of his/her RLS sensory symptoms, (ii) a signaling system that interfaces with the device and periodically alerts the user to assess his/her RLS sensory symptoms and enter or input a rating into the device, and (iii) a sensing system that allows one to determine a patient's compliance with prescribed test conditions and the environmental conditions surround the patent at the time of the periodic assessments, and (iv) a sensing system that detects and records RLS-impacted motor signs or movements of the wearer than can provide something of an objective measure of the RLS phenomena that a wearer experiences. See FIG. 1

The portability of this SHIRE monitor makes possible this invention's unique RLS monitoring methodology that includes what are hereinafter referred to as “Home-based Suggested Immobilization Test (H-SIT)” sessions. Test standards and databases are developed using this new methodology that permit it to be used to diagnose RLS and assess RLS severity.

The portability of the SHIRE monitor and its ease of use means that patients need no longer come to clinical facilities to have their RLS symptoms measured and analyzed. H-SIT sessions or tests can now conveniently be given multiple times per day, thereby making it possible to more easily investigate the previously reported circadian variations in RLS symptoms and the various or to-be-investigated criteria (e.g., (a) patient rest and/or activity, (b) day-to-day variations, (c) time of day, (d) treatment programs) that are thought to or may impact the severity of these symptoms.

More precisely, a SHIRE monitor, 10 includes: an 8 bit, on board programmable flash microcontroller 12, a switch array 14 that allows a user to input into the monitor the user's level of discomfort (LOD) due to RLS sensory symptoms (e.g., a 16 switch array (arranged vertically) arranged as follows: bottom switch=no discomfort, top Switch=severe discomfort), a corresponding indicator array 16 (e.g., LED indicators that are arranged vertically and located beside the switch array and which display the user's level of discomfort currently inputted or selected), a test START/STOP means or pushbutton switch 18 that starts & stops the monitor's internal timing device or clock (e.g., ±2 ppm real time clock (RTC) for accurate recording of event date and time stamping of incoming data) that is used to provide the “time” at which a user inputs each of the user's LOD observations, an EDIT pushbutton switch 20 that provides the opportunity for a user to re-enter the user's last LOD observation or selection.

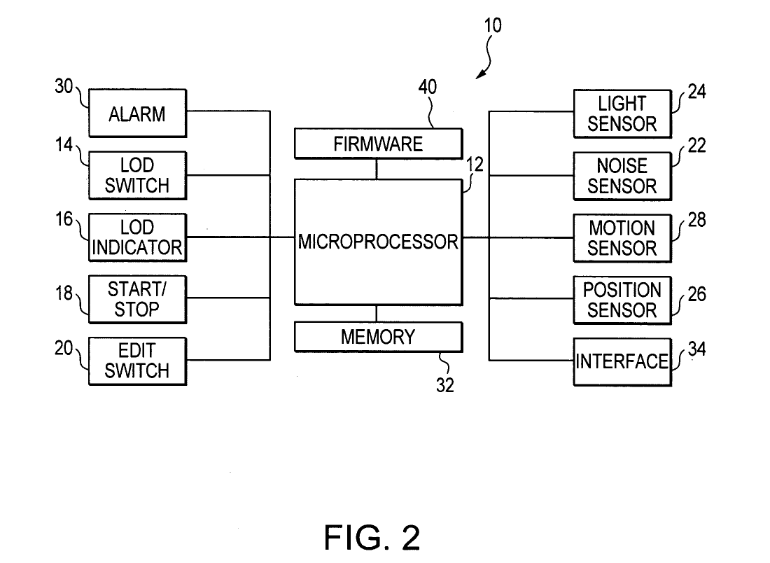

This device further includes an ambient audio (dB level) sensor 22 that is used to sense and allow for the subsequent periodic recording by the device of the general background sound level in the environment surrounding the user or patient during the administration of a RLS sensory symptoms monitoring test or evaluation period, an ambient light intensity (lux) sensor 24 that is used to similarly sense and allow for the subsequent periodic recording by the device of the general background light intensity levels in the environment surrounding the user during the test or evaluation period, a sensor or sensors 26 (e.g., a position, see U.S. Pat. No. 6,095,991 or inactivity sensor; alternatively, one could also use a video recorder) that may be located on a user's or wearer's extremities (e.g., wrist or leg) and configured to be attached to a position on the user's anatomy and is used to assess whether the user is complying with the prescribed protocol for his/her physical positioning (e.g., sitting up at a 45 degree angle in bed with one's feet stretched out horizontally and avoiding moving voluntarily) and inactivity, etc. during a test or evaluation period, a sensor 28 (e.g., a ±1.5-6.0 g accelerometer; a triaxial package in which only one channel is logged and which has a frequency response 0-40 Hz and is mated with an A/D convertor) that detects movements (e.g., PLM) of the wearer than can provide something of an objective measure of the RLS phenomena that a wearer experiences, an alarm (e.g., audio or visual) 30 that signals for a user that it is time for the user to assess and provide an input into the monitor of the user's LOD at the time of the alarm, a memory element 32 (e.g., 64 Kbyte EEPROM, nonvolatile memory) that has the capacity to store a specified amount (e.g., 10+ day recording capacity) of the user's LOD observations and specified data that characterizes the background environment in which the observations are being made, and a data interface 34 (e.g., USB 2.0) for downloading the recorded data into a personal computer for later analysis and/or long-term storage, and a power source (e.g., single AA alkaline battery) for the monitor. See FIG. 2.

The firmware 40 of the present invention utilizes structured programming implemented in C and assembly language. It is interrupt driven firmware and provides for: (a) timing function, (b) LOD switch array detection, (c) USB port data reception, (d) command setting with host computer, (e) synchronization of start of recording time with host computer (upon command), (f) delayed activation of front panel switches (LOD array, START, STOP, EDIT) so as to prevent false activations, (g) LOD switch-array averaging, (h) the subject pressing the START switch to begin each test (e.g., 1 hour H-SIT) on the first day of a multiple day and multi-test/day evaluation period (e.g., five 1 hour H-SIT tests/day over a 5 day period), (i) after a test is started, the alarming of the monitor at prescribed intervals (e.g., every 5 minutes), when it is time to for a user to assess and enter or select the user's next LOD assessment, (j) data input editing, if the user or subject feels that the wrong LOD entry was made, the user can use the EDIT switch to repeat the last entry, (k) notification of when during the day a test is to be conducted (e.g., if the subject fails to start a H-SIT test within 30 minutes of the previous day's time, the monitor initiates an alarm), (l) early termination, via the STOP switch, of a test if the user's discomfort level becomes unbearable, and (m) sequential memory storage of data blocks (e.g., a data block (625 bytes in size) is defined as the data contained in a 5 minute interval ending with the recording of a LOD entry; data block components: ambient audio (dB) level 300 bytes (1 byte/second), ambient light (lux) level 300 bytes (1 byte/second), right and left leg position 10 bytes (1 byte/30 seconds), LOD entry 1 byte (1 byte/5 minutes), LOD date and time stamp 5 bytes (5 bytes/5 minutes), H-SIT start date/time stamp 5 bytes, spare bytes−4 bytes; total=625 bytes per data block).

Another preferred embodiment of the present invention involves the methodology surrounding the use of this SHIRE monitor. This methodology includes having a patient utilize the SHIRE monitor while assuming a prescribed or standardized position (e.g., sitting upright in bed with legs stretched out horizontally). There should be no distracting stimuli in the room. After starting the SHIRE, the patient remains relaxed with as little movement as possible for a specified time period (e.g., 60 minutes). The patient is asked to rate the degree of leg discomfort on the SHIRE monitor at specific time intervals (e.g., 5 minutes) which are indicated by a signal from the monitor.

Since some RLS patients cannot survive resting for extended periods (e.g., sixty minutes) without getting up and moving, an option is provided that allows them to discontinue the test if it becomes intolerable. The duration of the test then becomes one of the measures that can be examined in evaluating a patient's condition.

The subject can be asked to repeat the test on consecutive nights (e.g., 5 nights) at about the same time period as for the initial night. Key measurements to be monitored and assessed with this sort of testing include: the average of the recorded sensory symptom severity (LOD) scores, the rate of increase of this score with increasing duration of rest, and the duration of the test if it was terminated early. The repeated testing over several nights and the ease of repeating the evaluation at various times permits quantitative assessments of the variability of the actual RLS symptoms under controlled conditions.

The present invention's SHIRE monitor or device is one component of a SHIRE Monitoring System which also includes a MS Windows based Interface Software and a USB cable for connecting the SHIRE device to a computer running this software. The general specifications for this Interface Software include: (a) developed using Microsoft Visual Basic 6, (b) runs on a Microsoft Windows based computer, (c) initialization and downloading of the SHIRE device is via a computer USB 2.0 interface, (d) initialization includes subject identification entry, comments entry, and a button switch to set the SHIRE's real time clock to the computer's date and time, (e) exports a comma delimited, .CSV format file of the data downloaded from the SHIRE device (this file can then be imported directly into Microsoft Excel), and (f) exported files display LOD entries in a 0-100 point scale, 0=no discomfort, 100=severe discomfort.

The foregoing is considered as illustrative only of the principles of the invention. Further, since numerous modifications and changes will readily occur to those skilled in the art, and because of the wide extent of the teachings disclosed herein, the foregoing disclosure should not be considered to limit the invention to the exact construction and operation shown and described herein. Accordingly, all suitable modifications and equivalents of the present disclosure may be resorted to and still considered to fall within the scope of the invention as are hereinafter set forth in claims to the present invention.

I claim:

1. A RLS sensory symptoms monitoring device for recording and analyzing, with the use of a personal computer, the RLS sensory symptoms of one wearing said device during a specified test period in which said wearer complies with a given test protocol, said device comprising:
a means for said wearer to input, at prescribed time intervals during said test period, into said device said wearer's assessment at said time intervals of said wearer's RLS sensory symptoms,
a means for indicating to said wearer the instances of said prescribed times during said period when said assessments are to be made and inputted into said device,
a means for sensing and inputting into said device data that measures the compliance of said wearer with said test protocol,
a microprocessor connected to said means, said microprocessor having embedded programmable memory,
a means for storing said inputted assessments and data,
a firmware means for controlling the operation of said microprocessor to control said indicating means and to temporarily store said assessments and data in said storage means, and
a means for transferring said stored assessments and data to said personal computer.

2. The monitoring device as recited in claim 1, further comprising a means for sensing and inputting into said device data that measures a RLS-impacted motor sign from said wearer.

3. The monitoring device as recited in claim 1, wherein said compliance sensing means including a sensor chosen from the group consisting of a position or motion or activity sensor configured so as to allow said sensor to be attached at a position on said wearer's anatomy, a video recording means configured for monitoring said wearer, or an environmental sensor that is configured to sense an aspect of the environment surrounding said wearer during said test.

4. The monitoring device as recited in claim 2, wherein said compliance sensing means including a sensor chosen from the group consisting of a position or motion or activity sensor configured so as to allow said sensor to be attached at a position on said wearer's anatomy, a video recording means configured for monitoring said wearer, or an environmental sensor that is configured to sense an aspect of the environment surrounding said wearer during said test.

5. The monitoring device as recited in claim 1, further comprising a software means for controlling the operation of said personal computer to analyze said transferred assessments and data.

6. The monitoring device as recited in claim 2, further comprising a software means for controlling the operation of said personal computer to analyze said transferred assessments and data.

7. The monitoring device as recited in claim 3, further comprising a software means for controlling the operation of said personal computer to analyze said transferred assessments and data.

8. The monitoring device as recited in claim 4, further comprising a software means for controlling the operation of said personal computer to analyze said transferred assessments and data.

9. The monitoring device as recited in claim 5, wherein said software means is configured to provide data analysis that includes computation of RLS symptom characterizing parameters chosen from the group of parameters including the average over a specified time period of the inputted assessments of said wearer's RLS sensory symptoms and the rate of change of said average in response to-be-investigated criteria that may impact the severity of these symptoms.

10. The monitoring device as recited in claim 8, wherein said software means is configured to provide data analysis that includes computation of RLS symptom characterizing parameters chosen from the group of parameters including the average over a specified time period of the inputted assessments of said wearer's RLS sensory symptoms and the rate of change of said average in response to-be-investigated criteria that may impact the severity of these symptoms.

11. A method for monitoring, recording and analyzing, with the use of a personal computer, the RLS sensory symptoms of an individual, said method comprising the steps of:
imposing on said individual, during a specified test period, a given test protocol that is to be complied with by said individual,
utilizing a monitoring device into which to input, at prescribed time intervals during said test period, said individual's assessment at said time intervals of said individual's RLS sensory symptoms,
indicating to said individual the instances of said prescribed times during said period when said assessments are to be made and inputted into said device,
sensing and inputting into said device data that measures the compliance with said test protocol,
storing said inputted assessments and sensed data,
utilizing a microprocessor within said device to control said indications, sensing and storage of data and assessments, and
transferring said stored assessments and data to said personal computer.

12. The method recited in claim 11, further comprising the step of sensing and inputting into said device data that measures a RLS-impacted motor sign from said wearer.

13. The method recited in claim 11, further comprising the step of using a software means for controlling the operation of said personal computer to analyze said transferred assessments and data.

14. The method recited in claim 12, further comprising the step of using a software means for controlling the operation of said personal computer to analyze said transferred assessments and data.

15. The method recited in claim 11, wherein said sensing step utilizes a sensor chosen from the group consisting of a position or motion or activity sensor configured so as to allow said sensor to be attached at a position on said wearer's anatomy, a video recording means configured for monitoring said wearer, or an environmental sensor that is configured to sense an aspect of the environment surrounding said wearer during said test.

16. The method recited in claim 13, wherein said sensing step utilizes a sensor chosen from the group consisting of a position or motion or activity sensor configured so as to allow said sensor to be attached at a position on said wearer's anatomy, a video recording means configured for monitoring said wearer, or an environmental sensor that is configured to sense an aspect of the environment surrounding said wearer during said test.

17. The method recited in claim 15, wherein said software means is configured to provide data analysis that includes computation of RLS symptom characterizing parameters chosen from the group of parameters including the average over a specified time period of the inputted assessments of said individual's RLS sensory symptoms and the rate of change of said average in response to-be-investigated criteria that may impact the severity of these symptoms.

18. The method recited in claim 16, wherein said software means is configured to provide data analysis that includes computation of RLS symptom characterizing parameters chosen from the group of parameters including the average over a specified time period of the inputted assessments of said individual's RLS sensory symptoms and the rate of change of said average in response to-be-investigated criteria that may impact the severity of these symptoms.
